(19) United States
(12) Patent Application Publication
(10) Pub. No.: US 2025/0270389 A1
(43) Pub. Date: Aug. 28, 2025

(54) STRETCHABLE SELF-HEALING HYDROGEL

(71) Applicant: IUCF-HYU (INDUSTRY-UNIVERSITY COOPERATION FOUNDATION HANYANG UNIVERSITY), Seoul (KR)

(72) Inventors: Kuen Yong LEE, Seoul (KR); Hyun Seung KIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/853,402

(22) PCT Filed: Mar. 30, 2023
(86) PCT No.: PCT/KR2023/004300

§ 371 Date: May 15, 2025

(30) Foreign Application Priority Data
Apr. 1, 2022 (KR) 10-2022-0041013

(51) Int. Cl. C08L 5/08; A61K 45/06; B33Y 70/00; C08K 5/25

(52) U.S. Cl. CPC C08L 5/08; A61K 45/06; C08K 5/25; B33Y 70/00; C08L 2312/00

(57) ABSTRACT

The present invention relates to a stretchable self-healing hydrogel. The hydrogel has excellent mechanical properties and stability, and stretchable and self-healing properties, and can be useful as a hydrogel for delivery of a drug or a cell, and a composition for 3D bioprinters.

### TECHNICAL FIELD

The present invention relates to a stretchable self-healing hydrogel.

### BACKGROUND ART

There are many efforts to replace damaged tissues or organs. Artificial substitutes and non-living tissues or organ transplants from animals have been typically used for patient treatment. However, they may not be a fundamental solution due to their heterogeneous composition compared to the original tissues or organs. Therefore, tissue engineering has recently emerged as an alternative to traditional surgical procedures. For example, in vitro cultured tissues or organs and cell delivery using biomaterials are being applied in tissue engineering approaches, and among various types of biomaterials, hydrogels are used very effectively.

Hydrogel is also called a hydrous gel and is a material that has a network structure in which water-soluble polymers form 3D crosslinks through physical bonds (hydrogen bonds, van der Waals force, hydrophobic interactions, etc.) or chemical bonds (covalent bonds) and is capable of containing a significant amount of water without being dissolved in an aqueous environment. Since hydrogels may be made from various water-soluble polymers, they have various chemical compositions and properties. In addition, they have high biocompatibility due to their high-water content and physicochemical similarity with the extracellular matrix. Due to these properties, hydrogels have drawn attention as one of the most attractive materials for medical and pharmacological applications. In particular, when hydrogels containing cells or drugs are injected, the self-healing characteristics of hydrogels are crucial to repair cracking caused by shear force.

Korean Patent Registration No. 10-1865168 discloses a self-healing hydrogel based on oxidized hyaluronate and a use thereof for delivering bioactive substances. However, the self-healing hydrogel has a problem in that it has low mechanical strength and thus is unable to maintain its shape or structure for a long time under physiological conditions.

### DISCLOSURE

### Technical Problem

In the above-described situation, the present inventors studied a hydrogel having strong mechanical properties with stretchability and self-healing properties, and confirmed that when an aqueous solution prepared by dissolving hydrazide-hyaluronic acid (hHA) and adipic acid dihydrazide is mixed with an aqueous solution prepared by dissolving oxidized hyaluronic acid (oHA), hHA and oHA are dual crosslinked through electrostatic interaction and chemical bonding to form a stretchable hydrogel. In addition, it was confirmed that the hydrogel has self-healing properties because adipic acid dihydrazide induces a competitive reaction, and the mechanical properties of the hydrogel are also improved by using ultra-high molecular weight hyaluronic acid.

Therefore, an object of the present invention is to provide a stretchable self-healing hydrogel and a method of preparing the same.

### Technical Solution

To achieve the object, one aspect of the present invention provides a stretchable self-healing hydrogel composition including:
oxidized hyaluronic acid (oHA); hydrazide-hyaluronic acid (hHA); and adipic acid dihydrazide (ADH),
wherein the hHA is a structure in which the ADH is covalently bonded to a hyaluronic acid (HA) chain,
an aldehyde group of the oHA forms a covalent bond with a hydrazide group of the hHA,
a carboxyl group of the oHA forms an ionic bond with a hydrazide group of the hHA, and
the oHA competitively reacts with the ADH and the hHA.

The term “hydrogel” as used herein refers to a 3D structure of hydrophilic polymers retaining a sufficient amount of water, and a “stretchable self-healing hydrogel” refers to a hydrogel exhibiting both stretchability and self-healing properties. The “stretchable self-healing hydrogel composition” refers to a composition that may be used to prepare a hydrogel exhibiting both stretchability and self-healing properties.

In the present specification, oHA refers to an HA derivative formed by oxidizing a diol group of HA to an aldehyde group, and the degree of oxidation is determined according to the number of aldehyde groups generated.

According to one embodiment of the present invention, the degree of oxidization of the oHA may be 10% to 50%, preferably 20% to 40%, and most preferably 34%.

In the hydrogel composition of the present invention, the properties of the hydrogel may be adjusted by adjusting the degree of oxidation of oHA. As the degree of oxidation increases, the aldehyde groups increase, and accordingly, the bonds with hHA increase, so that the properties of the hydrogel may be adjusted.

In the present invention, the hHA refers to a hyaluronic acid derivative in which adipic acid dihydrazide is bonded to HA via a carbodiimide bond, and a positively charged hydrazide group is introduced into hyaluronic acid, thereby increasing the positive charge of the hyaluronic acid.

According to one embodiment of the present invention, hHA may have a degree of substitution of 20% to 70%, preferably 20% to 60%, and most preferably 30% to 50%.

The degree of substitution refers to the number of adipic acid dihydrazides covalently bonded per 100 repeating units of hyaluronic acid, and is expressed as percentage (%) in the present invention. According to one embodiment of the present invention, the degree of substitution affects the stiffness of the hydrogel, and as the degree of substitution increases, the storage modulus of the hydrogel increases (FIG. 6). However, since a high degree of substitution exhibits cytotoxicity (FIG. 5), a degree of substitution in the above-described range is suitable for preparing a hydrogel.

According to one embodiment of the present invention, the molecular weight of the hHA also affects the stiffness of the hydrogel. Specifically, the hHA may have a weight-average molecular weight of 1×10⁵ to 20×10⁶ g/mol, preferably 1×10⁵ to 10×10⁶ g/mol, and more preferably 1.5×10⁵ to 2×10⁶ g/mol. The above-described molecular weight range is suitable because it is difficult to prepare a homogeneous hHA solution when the molecular weight is too high.

In the present invention, the oHA and the hHA may be included in the composition in a ratio of 1 to 5:0.1 to 5 (wt/wt), preferably 1 to 4:0.1 to 3 (wt/wt), but this range may vary depending on the degree of oxidation of oHA, the molecular weight and the degree of substitution of hHA.

In addition, based on the total weight of the composition, the hHA may be included in an amount of 0.1% to 5% by weight, preferably 1% to 4% by weight based on the total weight of the composition, but this range may vary depending on the molecular weight and the degree of substitution of hHA.

Adipic acid dihydrazide included in the composition imparts self-healing properties to the hydrogel through a competitive reaction, and specifically forms an imine bond by a Schiff base reaction with oHA.

The adipic acid dihydrazide may be included in an amount of 0.01% to 1% by weight, preferably 0.05% to 0.5% by weight, and more preferably 0.05% to 0.2% by weight based on the total weight of the composition. When the concentration of adipic acid dihydrazide is too low, self-healing properties are not exhibited, and when it is too high, the stiffness of the hydrogel decreases (FIG. 13).

Due to the properties of the above-described components, the stretchable self-healing hydrogel composition of the present invention is characterized in that oHA reacts with both hHA and ADH (free ADH). When cracking occurs and the crosslink between the components of the hydrogel is broken, the hHA and ADH competitively react with oHA again to form a crosslink, so that the hydrogel has self-healing properties.

In addition, the dual crosslinking between hHA and oHA through an electrostatic interaction (ionic bond) and chemical bonding (covalent bond) serves to improve the stretchability of the hydrogel. When stretching occurs, the chemical bonding is maintained while the electrostatic interaction acts reversibly, so the hydrogel exhibits stretchability.

Another aspect of the present invention provides a composition for 3D bioprinting, including the stretchable self-healing hydrogel composition.

In the present invention, the composition for 3D bioprinting refers to a material that may be used as an ink for a 3D bioprinter. The stretchable self-healing hydrogel composition of the present invention has self-healing properties when prepared into a hydrogel by adipic acid dihydrazide included in the composition. The hydrogel composition having self-healing properties may recover from cracking caused by a shear force when printed by a 3D bioprinter.

The present inventors used the stretchable self-healing hydrogel composition as an ink for a bioprinter to print a structure, and confirmed the stretchability and deformability of the structure, and also confirmed that the above properties were well maintained even after printing (FIG. 11).

Still another aspect of the present invention provides a method of preparing a stretchable self-healing hydrogel, including:
(a) mixing an ADH solution and hHA; and
(b) mixing an oHA solution with the mixture of (a).

The above method is a process of first mixing (+) polar substances (ADH and hHA) according to the polarity of the molecules to prevent ionic bonding prior to gelation, and then mixing the resulting mixture with a (−) polar substance (oHA). However, the method is not limited to the above-described mixing order, and a method of first mixing oHA with ADH or hHA and then mixing the remaining substance is also possible.

The description of oHA, hHA, and ADH is the same as the above description for the hydrogel composition. Since the stretchable self-healing hydrogel of the present invention has dual crosslinking between the components, a separate process for gelation of the hydrogel is not required.

Yet another aspect of the present invention provides a stretchable self-healing hydrogel prepared by the above method and a drug delivery system using the same.

The drug delivery system of the present invention may be prepared by a method including:
(a) mixing an ADH solution and hHA; and
(b) mixing an oHA solution and a target drug to be delivered with the mixture of (a).

The term “drug” as used herein refers to a substance that is capable of exhibiting a desired useful effect when introduced into the living body, and it may be selected from the group consisting of compounds, proteins, peptides, nucleic acids, saccharides, extracellular matrix substances, and cells.

In the present invention, the compound may be an antibiotic, an anticancer agent, an analgesic, an anti-inflammatory agent, an antiviral agent, an antibacterial agent, and the like, and the protein and peptide may be selected from the group consisting of hormones, cytokines, enzymes, antibodies, growth factors, transcriptional regulators, blood factors, vaccines, structural proteins, ligand proteins and receptors, cell surface antigens, and receptor antagonists. The nucleic acid may be an oligonucleotide, DNA, RNA, or peptide nucleic acid (PNA), and the saccharide may be selected from the group consisting of heparin, heparan sulfate, keratan sulfate, dermatan sulfate, chondroitin sulfate, and hyaluronate.

In addition, the extracellular matrix substance may be selected from the group consisting of collagen, fibronectin, gelatin, elastin, osteocalcin, fibrinogen, fibromodulin, tenascin, laminin, osteopontin, osteonectin, perlecan, versican, von Willebrand factor, and vitronectin, and the cell may be selected from the group consisting of fibroblasts, vascular endothelial cells, smooth muscle cells, nerve cells, bone cells, skin cells, chondrocytes, Schwann cells, and stem cells.

### Advantageous Effects

A stretchable self-healing hydrogel according to an embodiment of the present invention has excellent mechanical properties and stability along with stretchability and self-healing properties, so that it can be effectively used as a hydrogel for drug and cell delivery, and a composition for a 3D bioprinter.

### DESCRIPTION OF DRAWINGS

FIG. 1 briefly illustrates the principle by which a stretchable self-healing hydrogel according to an embodiment of the present invention exhibits self-healing properties.

FIG. 2 briefly illustrates the principle by which a stretchable self-healing hydrogel according to an example of the present invention exhibits stretchability.

FIG. 3 shows the Fourier-transform infrared spectroscopy (FT-IR) spectrum results of hyaluronic acid (HA), oxidized hyaluronic acid (oHA), hydrazide-hyaluronic acid (hHA), and an oHA/hHA hydrogel.

FIG. 4 shows the 1H NMR spectrum results of hHA with various degrees of substitution.

FIG. 5 shows the results of confirming cell viability after treating cells with various concentrations of oHA solutions with different degrees of substitution.

FIG. 6 shows the results of confirming the storage shear modulus of oHA/hHA hydrogels by varying the degree of substitution (A), the molecular weight of hHA and the oHA/hHA ratio (B), and the hHA1100 concentration (C).

FIG. 7A shows the results of confirming the stress-strain curves of oHA/hHA hydrogels prepared with hHA with different molecular weights.

FIG. 7B shows images of an oHA/hHA hydrogel before and after stretching.

FIG. 8A shows the results of confirming the complex viscosity of HA, hHA, and HA/hHA mixtures.

FIG. 8B shows the results of confirming the storage shear modulus (G′; filled symbols) and loss shear modulus (G″; open symbols) of HA, hHA, and HA/hHA mixtures: HA-black line; hHA-red line; and HA/hHA-blue line.

FIG. 9 shows the deformability of a stretchable self-healing (oHA/hHA/ADH) hydrogel according to an embodiment of the present invention.

FIG. 10 shows the results of confirming the self-healing behavior while applying a strain alternately to a stretchable self-healing (oHA/hHA/ADH) hydrogel.

FIG. 11 shows the results of confirming the stretchability (A) and self-healing behavior (B) of a 3D printed oHA/hHA hydrogel (−ADH/+P), an unprinted oHA/hHA/ADH hydrogel (+ADH/−P), and a 3D printed oHA/hHA/ADH hydrogel (+ADH/+P).

FIG. 12 shows the cell image (A) and the results of confirming cell viability (B) after printing the structure using the stretchable self-healing hydrogel containing cells as a bioink.

FIG. 13 shows the results of confirming the storage shear modulus of the gel after preparing the stretchable self-healing (oHA/hHA/ADH) hydrogel with different concentrations of ADH.

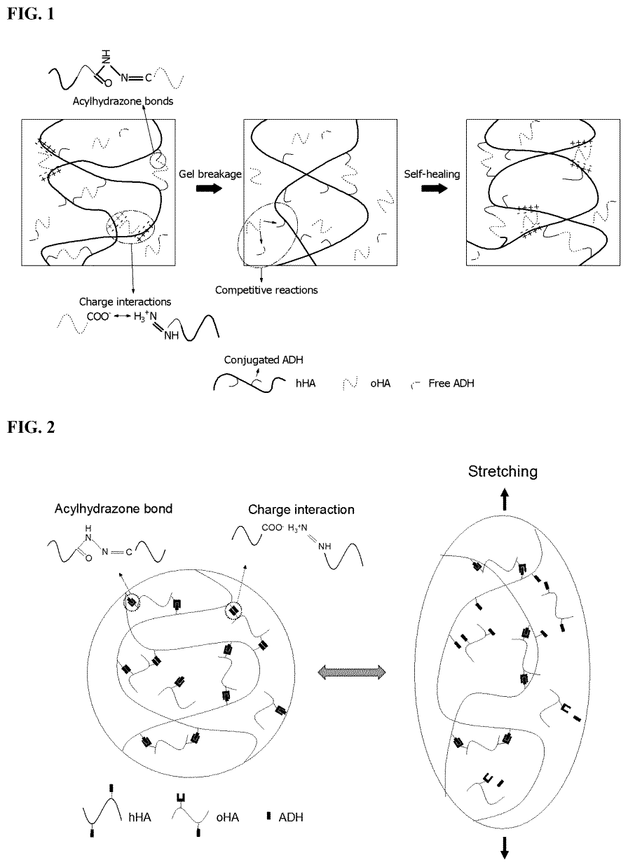

### MODES OF THE INVENTION

Below, one or more embodiments are described in more detail through examples. However, these examples are for illustrating one or more embodiments, and the scope of the present invention is not limited to these examples.

### Experimental Method

### 1. Reagents

Hyaluronic acid (HA) was purchased from Humedix (1000 kDa, B02-16-010; Anyang, South Korea) and Lifecore (200 kDa, 025841; 2000 kDa, 026489; Chaska, MN, USA). 1-Ethyl-3-(dimethylaminopropyl) carbodiimide (EDC) was purchased from Proteochem (Hurricane, UT, USA), and N-hydroxysulfosuccinimide sodium salt (sulfo-NHS) was purchased from Covachem (Loves Park, IL, USA).

Adipic acid dihydrazide (ADH), sodium periodate, 2-(N-morpholino) ethanesulfonic acid (MES) hydrate, transferrin human, and activated charcoal were purchased from Sigma Aldrich (St. Louis, MO, USA). Dulbecco's phosphate-buffered saline (DPBS), fetal bovine serum (FBS), Dulbecco's modified Eagle's medium nutrient mixture F-12 (DMEM/F-12), and penicillin-streptomycin were obtained from Gibco (Grand Island, NY, USA).

### 2. Preparation and Confirmation of oHA

1 g of HA was dissolved in 100 mL of deionized water overnight, and 0.26735 g of sodium periodate was added to the HA solution. After 24 hours, the reaction solution was dialyzed against distilled water for four days (molecular weight cut-off=3,500 g/mol) and filtered through a 0.22 μm filter. The synthesis of oHA was confirmed by Fourier transform infrared spectroscopy (FT-IR; Nicolet IS50, Thermo Fisher Scientific Inc.). HA and sodium periodate were allowed to react at various ratios to synthesize oHA with various degrees of oxidation.

The synthesized oHA samples and dry potassium bromide were ground together and compressed. Each resulting sample was scanned at a resolution of 4 cm⁻¹. The oxidation reaction of oHA was confirmed by 1H NMR spectrophotometry (VNMRS 600 MHz; Varian). D20 was used as a solvent, and the degree of oxidation of oHA was determined with 2,4,6-trinitrobenzene sulfonic acid (TNBS) (Kim et al., 2019).

### 3. Preparation and Confirmation of hHA

HA was dissolved in 0.1 M MES buffer (0.5 wt %, pH 6.0), and ADH, EDC, and sulfo-NHS were added at a molar ratio of 1:0.65:0.65 and allowed to react. On the next day, hHA was precipitated with ethanol (99.9%), and the precipitate was lyophilized. The synthesis of hHA was confirmed by Fourier-transform infrared (FT-IR) and ¹H NMR spectrophotometers. The reaction molar ratio of HA and ADH was varied to synthesize hHA with various degrees of substitution.

4. Size Exclusion Chromatography with Multi-Angle Laser Light Scattering (SEC-MALLS)

The molecular weight of HA derivatives (oHA and hHA) was calculated by SEC-MALLS (Shimadzu CO., LTD.). The SEC-MALLS system was equipped with a column (PL Aquagel-OH MIXED-H, 7.5 300 mm; Tosoh Bioscience), a multi-angle light scattering detector, and a differential refractometer (DAWN HELEOS II and Optilab Rex, Wyatt Technology Inc.).

Dextran was used as a standard polymer, PBS was used as a mobile phase, and the flow rate was set to 0.5 mL/min. The dn/dc value of the HA derivative was 0.167 mL/g, and the experiment was performed at 25° C. Data analysis was performed using ASTRA 6.1 software (Wyatt Technology Inc.).

### 5. Hydrogel Preparation and Confirmation of Properties

Hydrogels with stretchability were prepared by dissolving each of oHA and hHA in PBS and mixing the two resulting solutions. In addition, a hydrogel with self-healing ability and stretchability was prepared by mixing an ADH solution and hHA and then mixing an oHA acid solution therewith.

Hydrogel formation was confirmed by FT-IR spectrophotometry. The viscoelastic properties of the hydrogel were investigated at 5 Pa and 1 Hz using a rotational viscometer (Bohlin Gemini 150) equipped with a cone-and-plate fixture (20 mm diameter, 4° cone angle).

The stress-strain curve of the hydrogel was measured at a rate of 2.5 mm/min using Instron 5966 equipped with a 10 N load cell (Submersible Pneumatic Side Action Grips, Instron Inc.). The hydrogel samples (25 mm long, 8 mm wide, 1 mm thick) were prepared to fit the sample holder, and the sample holder was connected to the load cell.

### 6. 3D Printing

Various 3D structures were fabricated using a 3D printer (Invivo, Rokit Inc., South Korea). A self-healing oHA/hHA/ADH hydrogel was filled into a syringe equipped with a 25-gauge needle used as a nozzle. The motor pressure and fill density were kept constant at 300 N and 80%, respectively, and the printing speed was fixed at 300 mm/min.

### 7. Cell Culture and In Vitro Cell Viability Experiments

ATDC5 cells (RIKEN Cell Bank; Japan) used as model cells were cultured in a DMEM/F-12 medium containing 10% FBS and 1% PS under conditions of 37° C. and 5% CO₂. The cells were treated with each sample ([polymer]=0.01% to 0.5% by weight), an EZ-cytox solution (DoGen Bio CO., LTD., Korea) was added thereto, and the cells were incubated for four hours. Cell viability was confirmed by measuring the absorbance at 450 nm.

Hydrogels containing the ATDC5 cells at a concentration of 5×10⁶ cells/mL were printed in the shape of a disk (10 mm diameter, 1 mm thickness). The disks were cultured for three days under the conditions of 37° C. and 5% CO₂. Cell viability was assessed using the LIVE/DEAD Viability/Cytotoxicity kit (Invitrogen Inc., USA) according to the manufacturer's instructions. Cell images were taken using a confocal laser scanning microscope (TCS SP5; Leica Microsystems Inc., Germany).

### 8. Statistical Analysis

All data is presented as mean±standard deviation. Statistical analysis was performed using Student's t-test. A p-value of less than 0.05 or 0.01 was considered statistically significant (*p<0.05, ** p<0.01).

### Experimental Results

### 1. Preparation and Confirmation of oHA and hHA

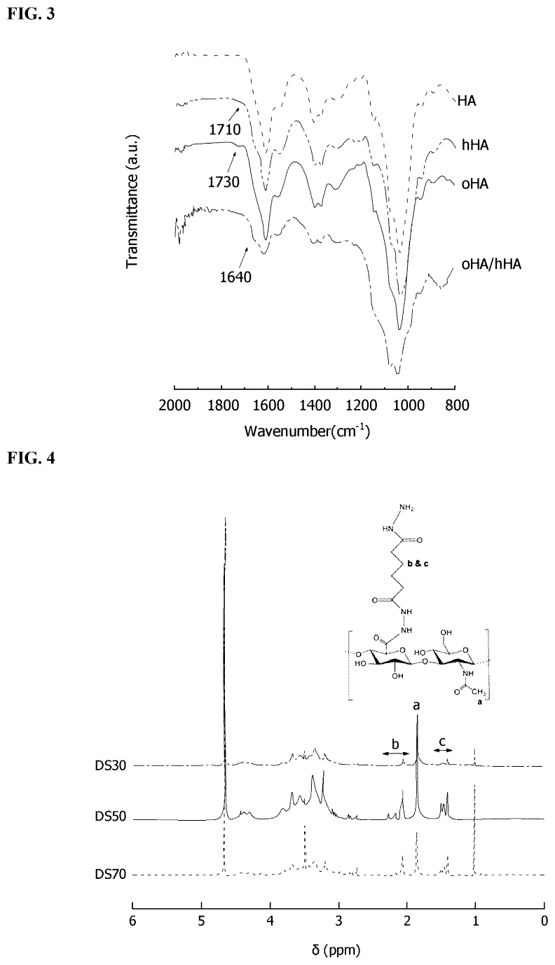

HA was partially oxidized with sodium periodate to prepare oHA having aldehyde groups. The oxidation reaction was confirmed by FT-IR and 1H NMR spectroscopy. The peak corresponding to the aldehyde group of oHA was observed at 1730 cm⁻¹ in the FT-IR spectrum (FIG. 3). New peaks at 4.5 ppm and 5.0 ppm in the 1H NMR spectrum support the formation of aldehyde groups in oHA (FIG. 4) (Park, Kim, Lee, & Lee, 2017). The number of aldehyde groups in oHA was determined by TNBS analysis (Kim et al., 2019). The degree of oxidation (%), defined as the number of oxidized units per 100 repeating units of HA, was calculated to be 34%.

HA was conjugated with ADH through a carbodiimide bond to form hHA. The conjugation with ADH was confirmed by FT-IR (FIG. 3). The peak corresponding to the amide group (C═O) in hHA was confirmed at 1710 cm⁻¹. The synthesis of hHA was also confirmed by 1H NMR spectroscopy (FIG. 4). A proton peak corresponding to ADH was observed in hHA (FIGS. 4B and 4C). The peak at 1.9 ppm corresponded to the acetamido moiety of the N-acetyl-D-glucosamine residue of HA (FIG. 4A). Various amounts of ADH was added to HA to synthesize hHAs with various degrees of substitution (DSs) ranging from 30 to 70%. The DS values were quantified by an 1H NMR spectrum (Table 1). The molecular weight of HA derivatives was determined by SEC-MALLS, and the results are shown in Table 1.

TABLE 1

| | | | Theoretical DS | Actual |
|---|---|---|---|---|
| Sample^{a} | Mw^{b} (g/mol) | R_{g, z}^{c} (nm) | (%) | DS (%) |
| oHA | 2.5 × 10³ | 25.8 | — | — |
| hHA210 | 2.1 × 10⁵ | 52.0 | — | — |
| hHA600 | 6.0 × 10⁵ | 105.3 | — | — |
| hHA1100 | 1.1 × 10⁶ | 138.5 | 30 | 28.5 |
| | | | 50 | 47.6 |
| | | | 70 | 67.5 |

^{a}The number following “hHA” indicates the weight-average molecular weight.
^{b}The weight-average molecular weight of hHA determined by SEC-MALLS measurement.
^{c}The Z-mean-square root-mean-square radius determined by SEC-MALLS measurement.

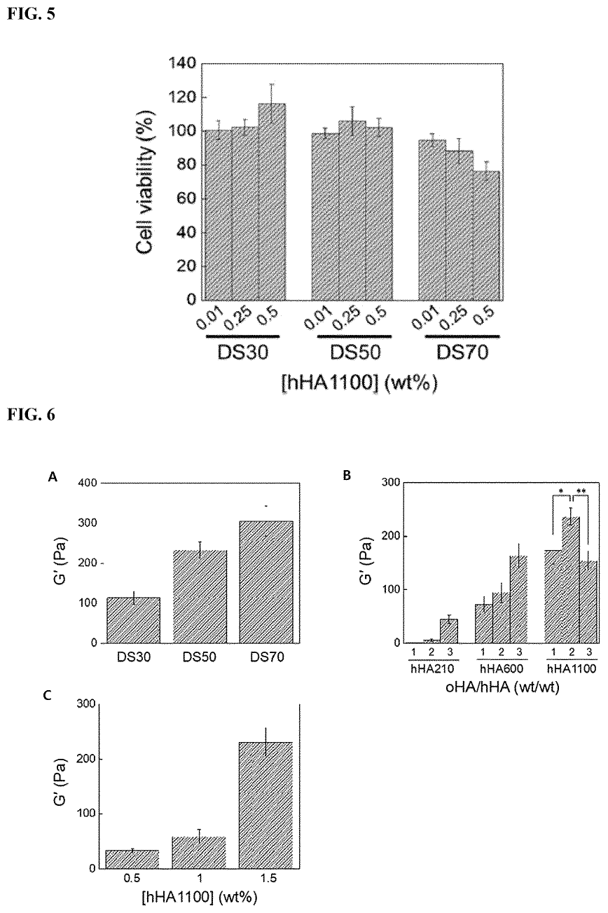

Next, the cytotoxicity of hHA was assessed in vitro using ATDC5 cells. Cell viability was not significantly affected by hHA1100-DS30 and hHA1100-DS50 at various polymer concentrations (FIG. 5). However, cell viability decreased as the concentration of hHA1100-DS70 increased, and the viability was less than 80% at [hHA1100-DS70]=0.5 wt %. These results may be due to the increase in the positive charges of hHA by the introduction of a positively charged hydrazide group into HA. Considering the cytotoxicity, it was concluded that hHA1100-DS70 was not suitable for further gel formation by adding oHA including cells.

2. Stretchable oHA/hHA Hydrogels

oHA/hHA hydrogels prepared without excipient crosslinking molecules were investigated. The aldehyde group of oHA and the hydrazide group of hHA may form a reversible acylhydrazone bond to form an oHA/hHA hydrogel. The formation of the acylhydrazone bond between oHA and hHA was confirmed by the disappearance of the aldehyde peak (1730 cm⁻¹) of oHA and the appearance of a new peak corresponding to the carbonyl band (1640 cm⁻¹) of the acylhydrazone bond in the FT-IR spectrum (FIG. 3) (Sun et al., 2019).

The effect of DS of hHA on hydrogel stiffness was confirmed using a rotational viscometer (FIG. 6). The molecular weight and polymer concentration of hHA were kept constant ([oHA]=3 wt %, [hHA1100]=1.5 wt %). The storage shear modulus (G′) of oHA/hHA hydrogels increased as the DS increased. However, hHA with DS70 showed cytotoxicity to ATDC5 cells (FIG. 5), so hHA with DS50 was selected and used in further experiments.

Next, the effect of hHA molecular weight on hydrogel stiffness was investigated. When the polymer concentration of the gel was the same, the G′ value increased as the molecular weight of hHA increased. When the hHA concentration was kept constant ([hHA]=1.5 wt %) and the oHA concentration was varied, the G′ value of the oHA/hHA hydrogel increased as the oHA concentration in the gel increased (FIG. 6). However, when the hydrogel was prepared using hHA1100 with [oHA]/[hHA]=3 (wt/wt), the G′ value decreased significantly. The very high molecular weight of hHA1100 may have hindered the bond formation with oHA under these experimental conditions.

Next, the concentration of hHA1100 was varied from 0.5 wt % to 1.5 wt % while maintaining the concentration of oHA at 3 wt %. Due to the large molecular weight of hHA1100, it was difficult to prepare a homogeneous hHA1100 solution at a concentration higher than 1.5 wt %. The hHA concentration in the oHA/hHA hydrogel significantly affected the G′ value of the gel (FIG. 6). Based on these results, the hydrogel prepared with [oHA]=3 wt % and [hHA1100-DS50]=1.5 wt % was selected for further study.

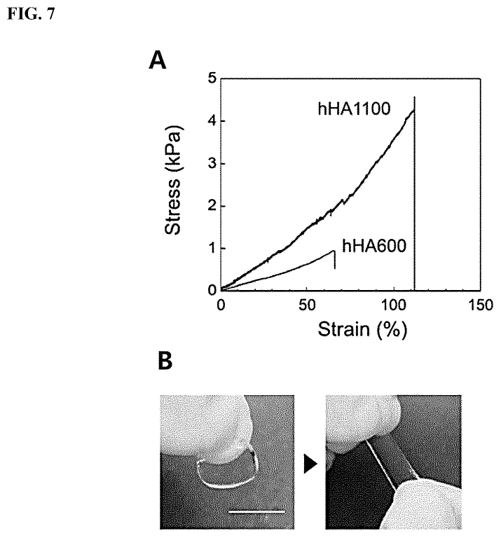

Next, a tensile test was performed on the oHA/hHA hydrogels. The molecular weight of hHA was varied while the polymer concentration was kept constant. The elongation at break varied depending on the molecular weight of hHA (FIG. 7). Interestingly, the oHA/hHA1100 hydrogel was able to stretch to more than twice its original length (FIG. 7).

On the other hand, conventional polysaccharide-based hydrogels are generally weak and brittle (Lee & Mooney, 2001). For example, HA/bacterial cellulose hydrogels and HA/silk fibroin hydrogels crosslinked with 1,4-butanediol diglycidyl ether may stretch to about 40% and 50% of their original lengths, respectively (Elia et al., 2013; Tang et al., 2021). HA/gelatin hydrogels may also stretch to 60% (Chang et al., 2021). Common stretchable hydrogel systems contain synthetic polymers such as polyacrylamide (PAAm). Alginate/PAAm hydrogels and acrylamide/poly(ethylene glycol) diacrylate hydrogels exhibited excellent stretchability (Ge et al., 2021; Sun et al., 2012).

The oHA/hHA hydrogels consisting solely of HA derivatives without the use of additional synthetic polymers may stretch up to approximately 2.1 times their original length, so they may find many useful biomedical applications, including those in tissue engineering.

Dual crosslinked hydrogels exhibit enhanced stretchability and toughness (Sun et al., 2012; Wu et al., 2018; Yang & Yuan, 2019). The dual crosslinked hydrogels are maintained by two different crosslinks. When stress is applied, a first crosslink is broken, and energy is dissipated. Then, a second crosslink maintains elasticity and is able to withstand greater stress (Chen et al., 2016; Zhang et al., 2018). Ionic crosslinking and covalent crosslinking are commonly used to prepare dual networks (Bakarich et al., 2013; Bakarich et al., 2012; Stevens, Calvert, & Wallace, 2013; Sun et al., 2012).

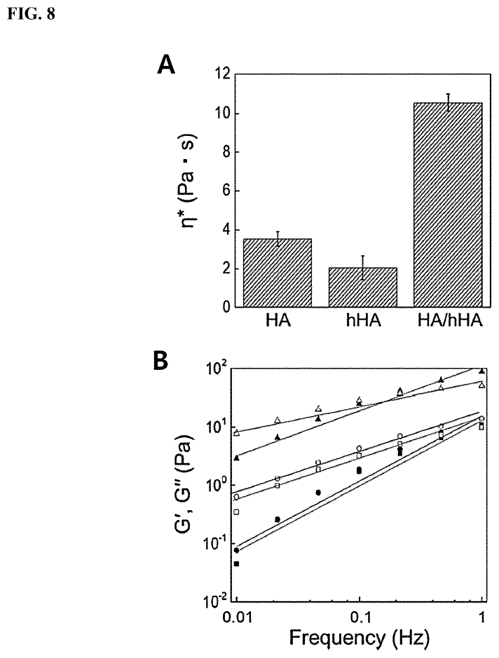

The improved stretchability of oHA/hHA hydrogels may also be attributed to the formation of dual crosslinks in the gels. Although HA is inherently negatively charged due to its carboxyl group (Jeon, Yoo & Park, 2015), the conjugation of ADH and HA may increase the positive charge due to the hydrazide group of ADH. The significant increase in the complex viscosity of the HA/hHA simple mixture compared to that of HA or hHA alone may be explained by the electrostatic interaction between HA and hHA (FIG. 8). In addition, the significant increase in the G′ value of the HA/hHA mixture and the intersection of the modulus curves at various frequencies prove that a gel-like structure is formed through ionic crosslinking between HA and hHA (FIG. 8).

When oHA/hHA hydrogels are stretched, the first bond (bond by electrostatic interaction) is broken, while the second bond (i.e., crosslink by acylhydrazone bond) is maintained, so that the hydrogels maintain elasticity. Therefore, both ionic bonds and covalent bonds in oHA/hHA hydrogels may improve the stretchability of the gels.

3. oHA/hHA/ADH Hydrogels with Self-Healing Ability and Stretchability

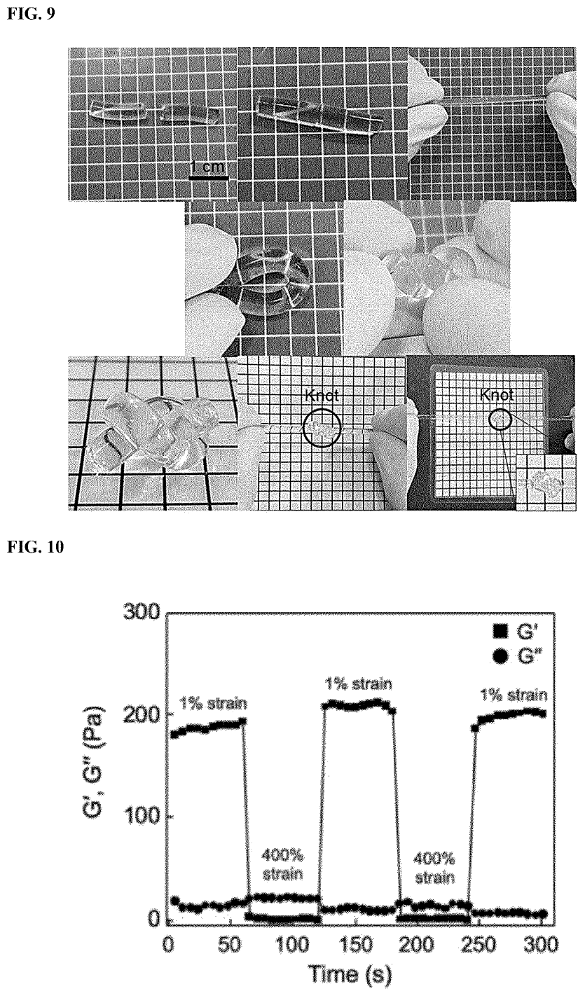

The self-healing ability of oHA/hHA/ADH hydrogels was investigated. The hydrogels were cut into two pieces, reattached, and manually stretched after 15 minutes. The self-healed hydrogels were able to stretch to about twice their original lengths without being broken. In addition, the oHA/hHA/ADH hydrogels were stretchable and thus could be formed into various shapes, and they were able to stretch while maintaining a bent, twisted, or knotted structure (FIG. 9).

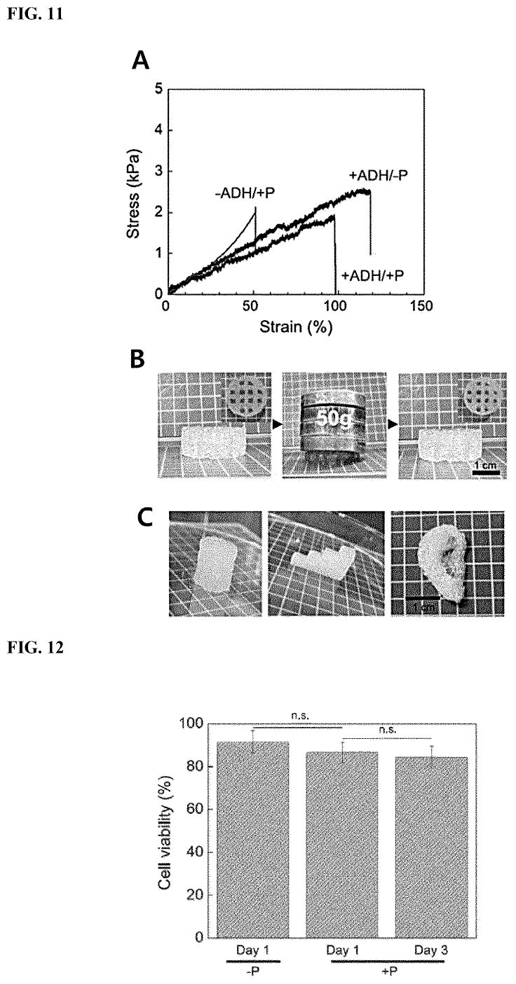

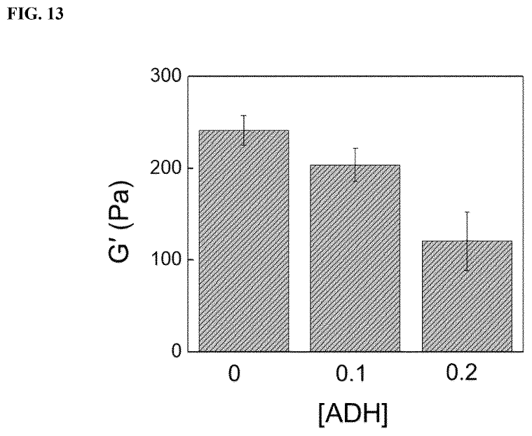

In addition, the self-healing properties of oHA/hHA/ADH hydrogels were further assessed using a rotational viscometer. The strain was varied from 1% to 400%, and it was confirmed that the G′ value of the gel was recovered when the high strain (400% strain) was removed (FIG. 10).

4. 3D Printing with oHA/hHA/ADH Hydrogels

3D structures were fabricated using oHA/hHA/ADH hydrogels. A tensile test was performed, and the results confirmed that there was no difference in stretchability between the oHA/hHA hydrogels (FIG. 7A) and the oHA/hHA/ADH hydrogel (+ADH/−P in FIG. 11), thereby confirming that the addition of ADH did not affect stretchability. On the other hand, the 3D printed oHA/hHA hydrogel (−ADH/+P) showed poor self-healing ability, so the elongation at break was reduced. However, the oHA/hHA/ADH hydrogels maintained their stretchability even after the printing process (+ADH/+P), which was nearly 90% of that of the unprinted gel (+ADH/−P) (FIG. 11).

Next, the high deformability of the 3D printed structure was tested. A mesh-shaped 3D structure was compressed with a weight, and when the weight was removed, the structure immediately recovered to its original shape (FIG. 11). In addition, various 3D structures could be fabricated using the oHA/hHA/ADH hydrogels (FIG. 11).

5. Confirmation of Cell Viability in oHA/hHA/ADH Hydrogels

ATDC5 cells were encapsulated in the oHA/hHA/ADH hydrogels, and their viability was assessed by a LIVE/DEAD assay. The results showed that printing (+P) did not affect cell viability. After three days of culture, about 85% of the cells survived within the printed oHA/hHA/ADH hydrogels (FIG. 12).

These results indicate that the 3D printable oHA/hHA/ADH hydrogel system may have potential in tissue engineering, including 3D printing of customized tissue constructs.

HA-based inks for bioprinting require additional crosslinking processes and have limited applications due to gel's inherent instability (brittleness). Methacrylated HA is a widely used bioink for 3D printing, which generally requires UV irradiation to form a gel. Alginate is also widely used in extrusion-based bioprinting. However, alginate also requires gelation using calcium ions after printing to form a solid structure (Mallakpour, Azadi, & Hussain, 2021; Piras & Smith, 2020).

On the other hand, the oHA/hHA/ADH hydrogels according to the present invention do not require an additional process to form a solid structure after 3D printing, which is advantageous in printing with biological materials such as proteins and cells.

Meanwhile, many natural polysaccharides have rigid backbones, and polysaccharide-based hydrogels are generally weak and brittle (Kumar et al., 2019; Xiao & Grinstaff, 2017). For example, alginate hydrogels crosslinked with calcium ions are brittle, so they may not be used as tissue replacements and may not be properly stretched (Ibrahim, Azam, & Amin, 2019; Drury, Dennis, & Mooney, 2004; Kunwar et al., 2019; Serrano-Aroca, Iskandar, & Deb, 2018).

However, oHA/hHA/ADH hydrogels have advantages of high physical strength because they include chemical crosslinks (i.e., reversible acylhydrazone bonding) and physical crosslinks (i.e., electrostatic interactions), which allow the HA-based hydrogels to overcome their inherent weaknesses.

### REFERENCES

An, H., Lee, J. W., Lee, H. J., Seo, Y., Park, H., & Lee, K. Y. (2018). Hyaluronate-alginate hybrid hydrogels modified with biomimetic peptides for controlling the chondrocyte phenotype. Carbohydrate Polymers, 197, 422-430.
Askari, M., Naniz, M. A., Kouhi, M., Saberi, A., Zolfagharian, A., & Bodaghi, M. (2021). Recent progress in extrusion 3D bioprinting of hydrogel biomaterials for tissue regeneration: a comprehensive review with focus on advanced fabrication techniques. Biomaterials Science, 9 (3), 535-573.
Bakarich, S. E., Beirne, S., Wallace, G. G., & Spinks, G. M. (2013). Extrusion printing of ionic-covalent entanglement hydrogels with high toughness. Journal of Materials Chemistry B, 1 (38), 4939-4946.
Bakarich, S. E., Pidcock, G. C., Balding, P., Stevens, L., & Calvert, P. (2012). Recovery from applied strain in interpenetrating polymer network hydrogels with ionic and covalent cross-links. Soft Matter, 8 (39), 9985-9988.
Bt Ibrahim, S. F., Azam, N. A. N. M., & Amin, K. A. M. (2019). Sodium alginate film: The effect of crosslinker on physical and mechanical properties. IOP Conference Series: Materials Science and Engineering (Vol. 509, p. 012063): IOP Publishing.
Chang, K.-C., Chen, W.-C., Chen, C.-H., Ko, C.-L., Liu, S.-M., & Chen, J.-C. (2021). Chemical cross-linking on gelatin-hyaluronan loaded with hinokitiol for the preparation of guided tissue regeneration hydrogel membranes with antibacterial and biocompatible properties. Materials Science and Engineering: C, 119, 111576.
Chang, R., Nam, J., & Sun, W. (2008). Effects of dispensing pressure and nozzle diameter on cell survival from solid freeform fabrication-based direct cell writing. Tissue Engineering Part A, 14 (1), 41-48.
Chen, Q., Yan, X., Zhu, L., Chen, H., Jiang, B., Wei, D., . . . . Zheng, J. (2016). Improvement of mechanical strength and fatigue resistance of double network hydrogels by ionic coordination interactions. Chemistry of Materials, 28 (16), 5710-5720.
Chimene, D., Kaunas, R., & Gaharwar, A. K. (2020). Hydrogel bioink reinforcement for additive manufacturing: a focused review of emerging strategies. Advanced Materials, 32 (1), 1902026.
Cui, X., Li, J., Hartanto, Y., Durham, M., Tang, J., Zhang, H., . . . . Woodfield, T. (2020). Advances in extrusion 3D bioprinting: a focus on multicomponent hydrogel-based bioinks. Advanced healthcare materials, 9 (15), 1901648.
Drury, J. L., Dennis, R. G., & Mooney, D. J. (2004). The tensile properties of alginate hydrogels. Biomaterials, 25 (16), 3187-3199.
Elia, R., Newhide, D. R., Pedevillano, P. D., Reiss, G. R., Firpo, M. A., Hsu, E. W., . . . . Peattie, R. A. (2013). Silk-hyaluronan-based composite hydrogels: A novel, securable vehicle for drug delivery. Journal of Biomaterials Applications, 27 (6), 749-762.
England, S., Rajaram, A., Schreyer, D. J., & Chen, X. (2017). Bioprinted fibrin-factor XIII-hyaluronate hydrogel scaffolds with encapsulated Schwann cells and their in vitro characterization for use in nerve regeneration. Bioprinting, 5, 1-9.
Fan, H., Hu, Y., Zhang, C., Li, X., Lv, R., Qin, L., & Zhu, R. (2006). Cartilage regeneration using mesenchymal stem cells and a PLGA-gelatin/chondroitin/hyaluronate hybrid scaffold. Biomaterials, 27 (26), 4573-4580.
Ge, Q., Chen, Z., Cheng, J., Zhang, B., Zhang, Y.-F., Li, H., . . . . Magdassi, S. (2021). 3D printing of highly stretchable hydrogel with diverse UV curable polymers. Science Advances, 7 (2), eaba4261.
Jang, T.-S., Jung, H.-D., Pan, H. M., Han, W. T., Chen, S., & Song, J. (2018). 3D printing of hydrogel composite systems: Recent advances in technology for tissue engineering. International Journal of Bioprinting, 4 (1).
Jeon, S., Yoo, C. Y., & Park, S. N. (2015). Improved stability and skin permeability of sodium hyaluronate-chitosan multilayered liposomes by Layer-by-Layer electrostatic deposition for quercetin delivery. Colloids and surfaces B: Biointerfaces, 129, 7-14.
Kesti, M., Müller, M., Becher, J., Schnabelrauch, M., D'Este, M., Eglin, D., & Zenobi-Wong, M. (2015). A versatile bioink for three-dimensional printing of cellular scaffolds based on thermally and photo-triggered tandem gelation. Acta Biomaterialia, 11, 162-172.
Kim, H., Jeong, H., Han, S., Beack, S., Hwang, B. W., Shin, M., . . . . Hahn, S. K. (2017). Hyaluronate and its derivatives for customized biomedical applications. Biomaterials, 123, 155-171.
Kim, H. S., Kim, C., & Lee, K. Y. (2022). Three-dimensional bioprinting of polysaccharide-based self-healing hydrogels with dual cross-linking. Journal of Biomedical Materials Research Part A, 110 (4), 761-772.
Kim, S. W., Kim, D. Y., Roh, H. H., Kim, H. S., Lee, J. W., & Lee, K. Y. (2019). Three-dimensional bioprinting of cell-laden constructs using polysaccharide-based self-healing hydrogels. Biomacromolecules, 20 (5), 1860-1866.
Kirchmajer, D. M., & Gorkin Iii, R. (2015). An overview of the suitability of hydrogel-forming polymers for extrusion-based 3D-printing. Journal of Materials Chemistry B, 3 (20), 4105-4117.
Kumar, A., Matari, I. A. I., Choi, H., Kim, A., Suk, Y. J., Kim, J. Y., & Han, S. S. (2019). Development of halloysite nanotube/carboxylated-cellulose nanocrystal-reinforced and ionically-crosslinked polysaccharide hydrogels. Materials Science and Engineering: C, 104, 109983.
Kunwar, P., Jannini, A. V. S., Xiong, Z., Ransbottom, M. J., Perkins, J. S., Henderson, J. H., . . . . Soman, P. (2019). High-resolution 3D printing of stretchable hydrogel structures using optical projection lithography. ACS Applied Materials & Interfaces, 12 (1), 1640-1649.
Kuzucu, M., Vera, G., Beaumont, M., Fischer, S., Wei, P., Shastri, V. P., & Forget, A. (2021). Extrusion-Based 3D Bioprinting of Gradients of Stiffness, Cell Density, and Immobilized Peptide Using Thermogelling Hydrogels. ACS Biomaterials Science & Engineering, 7 (6), 2192-2197.
Lee, K. Y., & Mooney, D. J. (2001). Hydrogels for tissue engineering. Chemical Reviews, 101 (7), 1869-1880.
Loebel, C., Rodell, C. B., Chen, M. H., & Burdick, J. A. (2017). Shear-thinning and self-healing hydrogels as injectable therapeutics and for 3D-printing. Nature Protocols, 12 (8), 1521-1541.
Mallakpour, S., Azadi, E., & Hussain, C. M. (2021). State-of-the-art of 3D printing technology of alginate-based hydrogels—an emerging technique for industrial applications. Advances in Colloid and Interface Science, 293, 102436.
Mobaraki, M., Ghaffari, M., Yazdanpanah, A., Luo, Y., & Mills, D. (2020). Bioinks and bioprinting: A focused review. Bioprinting, 18, e00080.
Moroni, L., Boland, T., Burdick, J. A., De Maria, C., Derby, B., Forgacs, G., . . . . Mironov, V. A. (2018). Biofabrication: a guide to technology and terminology. Trends in Biotechnology, 36 (4), 384-402.
Nadgorny, M., Xiao, Z., & Connal, L. A. (2017). 2D and 3D-printing of self-healing gels: design and extrusion of self-rolling objects. Molecular Systems Design & Engineering, 2 (3), 283-292.
Norioka, C., Inamoto, Y., Hajime, C., Kawamura, A., & Miyata, T. (2021). A universal method to easily design tough and stretchable hydrogels. NPG Asia Materials, 13 (1), 1-10.
Ouyang, L., Highley, C. B., Rodell, C. B., Sun, W., & Burdick, J. A. (2016). 3D printing of shear-thinning hyaluronic acid hydrogels with secondary cross-linking. ACS Biomaterials Science & Engineering, 2 (10), 1743-1751.
Park, H., Kim, S. W., Lee, J. W., & Lee, K. Y. (2017). Injectable hydrogels prepared from partially oxidized hyaluronate and glycol chitosan for chondrocyte encapsulation. Carbohydrate Polymers, 157, 1281-1287.
Park, H., Lee, H. J., An, H., & Lee, K. Y. (2017). Alginate hydrogels modified with low molecular weight hyaluronate for cartilage regeneration. Carbohydrate Polymers, 162, 100-107.
Park, H., Woo, E. K., & Lee, K. Y. (2014). Ionically cross-linkable hyaluronate-based hydrogels for injectable cell delivery. Journal of Controlled Release, 196, 146-153.
Petta, D., D'amora, U., Ambrosio, L., Grijpma, D., Eglin, D., & D'este, M. (2020). Hyaluronic acid as a bioink for extrusion-based 3D printing. Biofabrication, 12 (3), 032001.
Piras, C. C., & Smith, D. K. (2020). Multicomponent polysaccharide alginate-based bioinks. Journal of Materials Chemistry B, 8 (36), 8171-8188.
Puertas-Bartolomi, M., W³odarczyk-Biegun, M. K., Del Campo, A., Vazquez-Lasa, B., & San Roman, J. (2020). 3D printing of a reactive hydrogel bio-ink using a static mixing tool. Polymers, 12 (9), 1986.
Ramiah, P., Du Toit, L. C., Choonara, Y. E., Kondiah, P. P., & Pillay, V. (2020). Hydrogel-based bioinks for 3D bioprinting in tissue regeneration. Frontiers in Materials, 7, 76.
Serrano-Aroca., Iskandar, L., & Deb, S. (2018). Green synthetic routes to alginate-graphene oxide composite hydrogels with enhanced physical properties for bioengineering applications. European Polymer Journal, 103, 198-206.
Stevens, L., Calvert, P., & Wallace, G. G. (2013). Ionic-covalent entanglement hydrogels from gellan gum, carrageenan and an epoxy-amine. Soft Matter, 9 (11), 3009-3012.
Sun, C., Jia, H., Lei, K., Zhu, D., Gao, Y., Zheng, Z., & Wang, X. (2019). Self-healing hydrogels with stimuli responsiveness based on acylhydrazone bonds. Polymer, 160, 246-253.
Sun, J.-Y., Zhao, X., Illeperuma, W. R., Chaudhuri, O., Oh, K. H., Mooney, D. J., . . . . Suo, Z. (2012). Highly stretchable and tough hydrogels. Nature, 489 (7414), 133-136.
Tang, S., Chi, K., Xu, H., Yong, Q., Yang, J., & Catchmark, J. M. (2021). A covalently cross-linked hyaluronic acid/bacterial cellulose composite hydrogel for potential biological applications. Carbohydrate Polymers, 252, 117123.
Unagolla, J. M., & Jayasuriya, A. C. (2020). Hydrogel-based 3D bioprinting: A comprehensive review on cell-laden hydrogels, bioink formulations, and future perspectives. Applied Materials Today, 18, 100479.
Wu, J., Han, S., Yang, T., Li, Z., Wu, Z., Gui, X., . . . . Liu, C. (2018). Highly stretchable and transparent thermistor based on self-healing double network hydrogel. ACS Applied Materials & Interfaces, 10 (22), 19097-19105.
Xiao, R., & Grinstaff, M. W. (2017). Chemical synthesis of polysaccharides and polysaccharide mimetics. Progress in Polymer Science, 74, 78-116.
Yang, B., & Yuan, W. (2019). Highly stretchable and transparent double-network hydrogel ionic conductors as flexible thermal-mechanical dual sensors and electroluminescent devices. ACS Applied Materials & Interfaces, 11 (18), 16765-16775.
Zhang, H., Cong, Y., Osi, A. R., Zhou, Y., Huang, F., Zaccaria, R. P., . . . . Fu, J. (2020). Direct 3D printed biomimetic scaffolds based on hydrogel microparticles for cell spheroid growth. Advanced Functional Materials, 30 (13), 1910573.
Zhang, W., Liu, X., Wang, J., Tang, J., Hu, J., Lu, T., & Suo, Z. (2018). Fatigue of double-network hydrogels. Engineering Fracture Mechanics, 187, 74-93.

1. A stretchable self-healing hydrogel composition comprising oxidized hyaluronic acid, hydrazide-hyaluronic acid, and adipic acid dihydrazide,
wherein the hydrazide-hyaluronic acid is a structure in which the adipic acid dihydrazide is covalently bonded to a hyaluronic acid chain,
an aldehyde group of the oxidized hyaluronic acid forms a covalent bond with a hydrazide group of the hydrazide-hyaluronic acid,
a carboxyl group of the oxidized hyaluronic acid forms an ionic bond with a hydrazide group of the hydrazide-hyaluronic acid, and
the oxidized hyaluronic acid competitively reacts with the adipic acid dihydrazide and the hydrazide-hyaluronic acid.

2. The composition of claim 1, wherein the hydrazide-hyaluronic acid has a weight-average molecular weight of 1×10⁵ to 20×10⁶ g/mol.

3. The composition of claim 1, wherein the hydrazide-hyaluronic acid has a degree of substitution of 20% to 70%.

4. The composition of claim 1, wherein the oxidized hyaluronic acid and the hydrazide-hyaluronic acid are included in the composition at a ratio of 1 to 5:0.1 to 5 (wt/wt).

5. The composition of claim 1, wherein the hydrazide-hyaluronic acid is included in an amount of 0.1% to 5% by weight based on the total weight of the composition.

6. The composition of claim 1, wherein the oxidized hyaluronic acid has an oxidation degree of 10% to 50%.

7. A composition for three-dimensional bioprinting, comprising the stretchable self-healing hydrogel composition of claim 1.

8. A method of preparing a stretchable self-healing hydrogel, comprising:
(a) mixing an adipic acid dihydrazide solution and hydrazide-hyaluronic acid; and
(b) mixing an oxidized hyaluronic acid solution with the mixture of (a),
wherein the hydrazide-hyaluronic acid is a structure in which the adipic acid dihydrazide is covalently bonded to a hyaluronic acid chain,
an aldehyde group of the oxidized hyaluronic acid forms a covalent bond with a hydrazide group of the hydrazide-hyaluronic acid,
a carboxyl group of the oxidized hyaluronic acid forms an ionic bond with a hydrazide group of the hydrazide-hyaluronic acid, and
the oxidized hyaluronic acid competitively reacts with the adipic acid dihydrazide and the hydrazide-hyaluronic acid.

9. The method of claim 8, wherein the hydrazide-hyaluronic acid has a degree of substitution of 20% to 70%.

10. The method of claim 8, wherein the hydrazide-hyaluronic acid is included in an amount of 0.1% to 5% by weight based on the total weight of the hydrogel.

11. A stretchable self-healing hydrogel prepared by the method of claim 8.

12. A drug delivery system comprising the stretchable self-healing hydrogel of claim 11.

13. The drug delivery system of claim 12, wherein the drug is selected from the group consisting of compounds, proteins, peptides, nucleic acids, saccharides, and cells.

14. A stretchable hydrogel composition comprising oxidized hyaluronic acid and hydrazide-hyaluronic acid,
wherein the hydrazide-hyaluronic acid is a structure in which adipic acid dihydrazide is covalently bonded to a hyaluronic acid chain, and
an aldehyde group of the oxidized hyaluronic acid forms a covalent bond with a hydrazide group of the hydrazide-hyaluronic acid.
